(19) United States
(12) Patent Application Publication
(10) Pub. No.: US 2015/0160190 A1
(43) Pub. Date: Jun. 11, 2015

(54) VAPORIZED FLUID DETECTOR CALIBRATION DEVICE

(71) Applicant: Lifeloc Technologies, Inc., Wheat Ridge, CO (US)

(72) Inventor: Gurumurthi Ravishankar, Englewood, CO (US)

(21) Appl. No.: 14/559,498

(22) Filed: Dec. 3, 2014

Related U.S. Application Data
(60) Provisional application No. 61/912,284, filed on Dec. 5, 2013.
(60) Provisional application No. 61/913,173, filed on Dec. 6, 2013.

(51) Int. Cl. G01N 33/497

(52) U.S. Cl. CPC G01N 33/4972

(57) ABSTRACT

A disposable, single use calibration cartridge for calibrating vaporized fluid detection equipment, such as a breath alcohol tester. The calibration cartridge has a sealed housing having an interior volume, with a saturated working fluid present therein, the saturated working fluid present both in a vapor phase and a liquid phase. The working fluid includes the substance to be detected and a carrier fluid.

### CROSS-REFERENCE TO RELATED APPLICATIONS

This application claims priority to U.S. provisional application 61/912,284 filed Dec. 5, 2013 and to U.S. provisional application 61/913,173 filed Dec. 6, 2013, both of which are incorporated herein by reference in their entirety for all purposes.

### BACKGROUND

It is commonly accepted that for the purposes of public safety on public roads and elsewhere, individuals should not operate potentially dangerous machines (such as automobiles) under the influence of alcohol. As a result, many jurisdictions have enacted laws that impose fines or other criminal penalties if individuals are operating an automobile or performing other activities while having a blood alcohol content (BAC) that exceeds a certain threshold. Further, many workplaces have similar rules in place to prevent employees from performing their duties under the influence of alcohol.

In order to effectively enforce these law and rules, alcohol concentration in human breath is often used as a proxy for BAC and is compared against legal limits to determine an individual's compliance with an applicable law or rule. There are a variety of measuring instruments currently used for determining the concentration of alcohol in human breath (referred to herein as breath alcohol testers). However, since a determination that an individual's BAC is above the legal threshold can result in criminal penalties, loss of a job, or other sanctions against the individual, the accuracy of these breath alcohol testers is critical for consistent measurement of BAC and application of the laws or rules.

An individual's breath includes vaporizes fluids (e.g., water, alcohol, and other substances). Breath alcohol testers that measure vaporized alcohol concentration within the individual's breath vapor may be calibrated periodically against a standard with a known alcohol concentration within a gas (referred to herein as a calibration standard). Further, the breath alcohol testers may also be checked against the calibration standard periodically to ensure that no recalibration is needed or to prompt a recalibration if the breath alcohol tester is found to be outside of a permissible calibration range. Portable, accurate, and user-friendly calibration equipment encourages breath alcohol test administrators to ensure that the breath alcohol testers are correctly calibrated at all times.

### SUMMARY

The present description is directed to disposable (one-time-use) calibration cartridges, heaters/coolers for calibration cartridges, and methods of using the calibration cartridge to calibrate vaporized fluid detectors, such as volatile substance detectors. An example of a volatile substance detector is a breath alcohol detector.

One particular implementation described herein is a disposable calibration cartridge having a housing having an interior volume, a saturated working fluid vapor including a detected substance and a carrier fluid in the interior volume, and a sealing membrane on the housing forming a hermetically sealed interior volume.

Another particular implementation is a disposable calibration cartridge having a sealed housing having a vapor space compartment and a calibration medium compartment, a working fluid present in a vapor phase in the vapor space compartment, and the working fluid present in both a vapor phase and a liquid phase in the calibration medium compartment, and an access port through the housing into the vapor space compartment.

Yet another particular implementation is a temperature adjuster for a disposable calibration cartridge, the temperature adjuster having a receptacle to receive the disposable calibration cartridge at least partially therein, and having a heating mechanism to raise the temperature of the calibration cartridge to within 0.1 degree Celsius of a desired temperature. In other implementations, the temperature adjuster has a cooling mechanism to lower the temperature of the calibration cartridge to within 0.1 degree Celsius of a desired temperature.

Yet another particular implementation is a method of calibrating a fluid detector. The method includes accessing a saturated working fluid vapor in a calibration cartridge, the saturated working fluid vapor including a detected substance and a carrier fluid, and performing a calibration operation on the vaporized fluid detector using the saturated working fluid vapor within the calibration cartridge.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. These and various other features and advantages will be apparent from a reading of the following detailed description.

### BRIEF DESCRIPTION OF THE DRAWING

FIG. 1 is a schematic cross-sectional side view of an example disposable calibration cartridge.

FIG. 2 is a schematic cross-sectional side view of another example disposable calibration cartridge.

FIG. 3 is a schematic cross-sectional side view of another example disposable calibration cartridge.

FIG. 4 is a schematic cross-sectional side view of an example of a disposable calibration cartridge mounted to a cartridge heater.

FIG. 5 is a schematic cross-sectional side view of another example of a disposable calibration cartridge mounted to a cartridge heater.

FIG. 6A is a schematic side view and FIG. 6B is a schematic front view of an example of a disposable calibration cartridge mounted to a cartridge heater being used to calibrate a breath alcohol tester.

FIG. 7A is a schematic side view, FIG. 7B is a schematic front view, and FIG. 7C is a schematic top view of another example of a disposable calibration cartridge mounted to a cartridge heater being used to calibrate a breath alcohol tester.

FIG. 8 is a block diagram of example operations for using a disposable calibration cartridge to calibrate a vaporized fluid detector.

### DETAILED DESCRIPTION

Calibration standards for breath alcohol testers are generally of two types, wet bath and dry gas. A wet bath includes water vapor as a carrier gas while dry gas utilizes another carrier gas (e.g., nitrogen). The wet bath is prepared by combining a known proportion of ethanol and water in a partially filled open reservoir that is accurately heated to a desired temperature that approximates an individual's breath temperature (e.g., 34° C.). At equilibrium, gaseous headspace above the liquid water in the reservoir contains saturated gaseous-phase water with a known concentration of ethanol at the desired temperature. This known concentration of ethanol within the water vapor is used to calibrate and/or check the calibration of a breath alcohol tester. While accurate, wet baths are subject to splashing, tipping over, and only operate properly within a limited temperature range. Further, due to their relatively complex design, wet baths are not particularly easy or efficient to transport, which limits their use primarily to stationary controlled settings.

The dry gas method utilizes a carrier gas such as nitrogen or argon along with a known concentration of ethanol stored within a pressurized tank (e.g., with pressures ranging from 500-2500 psig). This known concentration of ethanol within the carrier gas is used to calibrate and/or check the calibration of a breath alcohol tester. Dry gas standards are provided in a variety of types of high-pressure cylinders typically containing 1 liter or more of pressurized gas. While dry gas standards are generally more portable that the wet bath, a 1-liter size pressurized bottle is still a significant burden to carry with a mobile breath alcohol tester. Further, if a dry gas standard has a leak, it is possible to lose a significant amount of calibration gas before a problem is noticed. Further still, some users (especially mobile ones) have concerns that even relatively small high-pressure gas tanks that, even while filled with generally nonflammable gas (e.g., nitrogen), are potentially dangerous due to their high storage pressure.

The presently disclosed technology combines aspects of the wet bath calibration standard with the portability of the dry gas standard to create new ultra-portable, safe, and easy to use calibration systems and associated methods. While the following description is specific to breath alcohol tester calibration systems, the disclosed calibration systems and associated methods may be applied to calibrate any volatile substance detector by using a calibration standard that incorporates the volatile substance to be detected. In other implementations, the disclosed calibration systems and associated methods may be applied to calibrate any non-volatile substance detector by using a calibration standard that incorporates the non-volatile substance to be detected in a liquid-phase/gaseous-phase equilibrium.

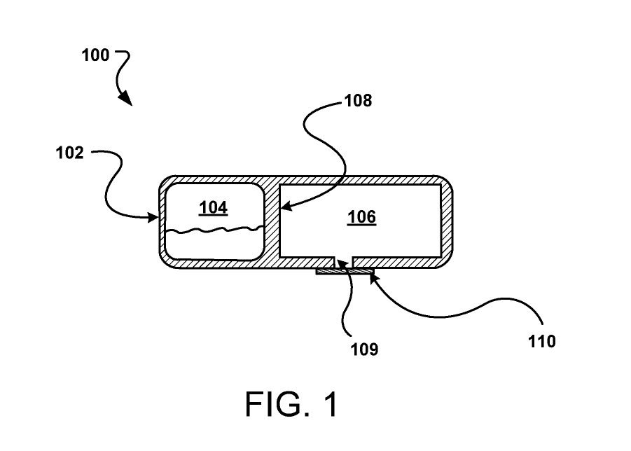

FIG. 1 illustrates an example disposable calibration cartridge 100. The cartridge 100 includes a housing 102 that encloses a calibration medium compartment 104 and a vapor space compartment 106, in this implementation, separated by a permeable barrier 108. A sampling port 109 is provided in the housing 102, in this implementation in a side wall of the housing 102, to provide access to the vapor space compartment 106. The sampling port 109 is covered by a sealing membrane 110 prior to use of the calibration cartridge 100, as shown in FIG. 1. Prior to use of the calibration cartridge 100, the housing 102 is sealed (e.g., a hermetic seal, or a seal impervious to gases) so internal fluids cannot escape the housing 102 and external fluids cannot enter the housing 102.

The calibration medium compartment 104 contains a precise ratio of a carrier fluid (e.g., water) and a volatile substance (e.g., alcohol, such as ethanol) existing in both liquid and gaseous phases as a saturated fluid within the housing 102. The precise carrier fluid/alcohol mixture is referred to herein as a “working fluid”. Ingredients other than the carrier fluid and the volatile substance are avoided in the working fluid. Further, the use of the term “fluid” herein explicitly refers to any non-solid phase of matter, including liquids and gases.

Fluids other than water may be used as the carrier medium so long as they can be combined with the desired volatile substance to form a homogeneous mixture and can be stored within the cartridge 100 at a liquid-phase/gaseous-phase equilibrium. In various implementations, the volatile substance concentration in the carrier fluid may range from 25 ppm to 1050 ppm, depending on the concentration of the volatile substance desired in the vapor phase. As an example, to achieve a vapor-phase 0.100 ethanol concentration (in water), the working fluid has 230-260 ppm ethanol in water depending on the expected partition ratio. As another example, to achieve a vapor-phase 0.080 ethanol concentration (in water), the working fluid has 190-208 ppm ethanol in water, and for a vapor-phase 0.040 ethanol concentration (in water), the working fluid has 95-105 ppm ethanol in water.

The calibration medium compartment 104 is illustrated as having an amount of liquid working fluid therein; not seen in the compartment 104 is the vaporous working fluid therein. Also, not seen in the vapor space 106 is the vaporous working fluid present therein.

The permeable barrier 108 allows passage of vapor phase working fluid therethrough, but inhibits the passage of liquid therethrough. The permeable barrier 108 may be a thin layer of perforated material or a series of layers that provide one or more convoluted pathways from the calibration medium compartment 104 to the vapor space compartment 106 that enables gaseous-phase working fluid transmission there through, but prevents or resists liquid-phase working fluid from being transmitted from the calibration medium compartment 104 to the vapor space compartment 106.

The vapor space compartment 106 contains gaseous-phase working fluid in relative equilibrium with the liquid-phase and vapor- or gaseous-phase working fluid within the calibration medium compartment 104. The sampling port 109 allows access to the gaseous-phase working fluid during a calibration operation. Prior to the calibration operation, the sealing membrane 110 seals the housing 102 as a fluid-tight closed environment where the working fluid is stored in equilibrium.

In some implementations, the sealing membrane 110 is a thin membrane, film or foil glued or otherwise attached to the housing 102 over the sampling port 109. Any membrane, film, foil or other element used for the sealing membrane 110 is non-liquid and non-vapor permeable. In other implementations, the sealing membrane 110 is merely a thin area in the housing 102 where the housing 102 is punctured to create the sampling port 109. In still other implementations, there is no sealing membrane and the sampling port is created by puncturing through the housing 102 in any convenient area. In still other implementations, the sealing membrane is a physically moveable element, such as a hinged cover over the sampling port 109 or a slideable door.

The housing 102 may have any suitable volume-enclosing shape and size. For example, the housing 102 may be cylindrical with dimensions ranging from 2.5 to 12.5 cm long and 0.25 to 2.5 cm in diameter. The housing 102 may have a total volume of 2 to 8 cubic centimeters, in some implementations 3 to 4 cubic centimeters. The vapor space compartment 106 may be of any volume greater than 0.1 or 0.2 cubic centimeters; examples volumes for the vapor space compartment 106 include 0.5 cubic cm, 1 cubic cm, and 2 cubic cm. The calibration medium compartment 104 may be of any volume greater than 0.1 or 0.2 cubic centimeters; examples volumes for the compartment 104 include 0.5 cubic cm, 1 cubic cm, 2 cubic cm, and 3 cubic cm. The combined volume of the calibration medium compartment 104 and the vapor space compartment 106 can be, for example, 1 to 10 cubic cm. In one particular example, the combined volume of the calibration medium compartment 104 and the vapor space compartment 106 is in the range of 5 to 6 cubic cm, and in another example is in the range of 5.5 to 6 cubic cm. The ratio of the vapor space compartment 106 to the calibration medium compartment 104 depends on the volatile substance and the desired volume of vapor at the desired concentration.

It is noted that the volume of vapor actually used to perform the calibration will vary depending on the device being calibrated. The volume of the vapor space compartment 106 is many multiples (e.g., 5×, 10×, 20×) of the actual sampling volume used.

The housing 102 can be made of any convenient non-permeable materials including plastic(s), metal(s), composite(s) and fibrous material(s), optionally with a non-liquid and non-vapor permeable coating (e.g., paper with a wax coating). Injection molding or rotation molding of polymeric material (plastic(s)) is a suitable manner for making the housing 102. Examples of polymeric materials that are particularly suited for use cartridges 100 configured to retain ethanol/water mixtures include high density polyester (HDPE), low density polyester (LDPE), polypropylene, polycarbonate, and various blends including these polymeric materials.

The permeable barrier 108 may be made of any convenient permeable materials (e.g., perforated or porous plastics, metals, and/or fibrous materials). An example of a barrier 108 that is permeable to vapor but non-permeable to liquid is expanded polytetrafluoroethylene (PTFE).

In some implementations, the housing 102 includes a moisture detector that changes color if the interior of the housing 102 dries up (e.g., if there is a leak to atmosphere). The moisture detector may provide a visual signal (e.g., green if it contains sufficient moisture and red if it does not) to a user that the calibration cartridge 100 usable or not usable. In other implementations, the housing 102 includes a detector responsive to an element or compound commonly present in the atmosphere, but not present in the housing 102 (e.g., nitrogen, oxygen, and carbon dioxide) and provides a similar visual signal to the user that the calibration cartridge 100 is usable or not usable if that element or compound is detected within the housing 102. In some implementations, the housing 102 includes a storage temperature indicator that indicates if the cartridge 100 had been exposed to (e.g., stored at) temperatures exceeding those specified.

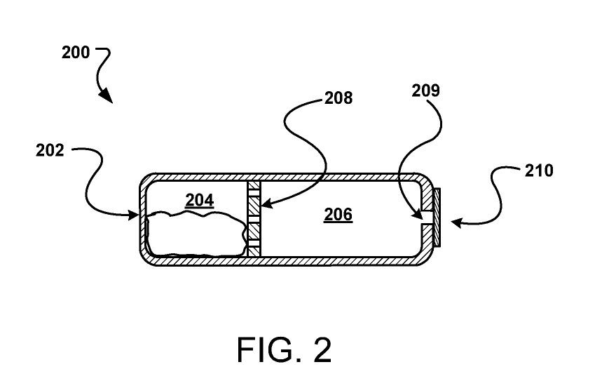

FIG. 2 illustrates another example disposable calibration cartridge 200. Various elements and features of cartridge 200 are the same or similar to like elements and features of cartridge 100 of FIG. 1, unless indicated otherwise.

The cartridge 200 includes a housing 202 that encloses a calibration medium compartment 204 and a vapor space compartment 206, in this implementation, separated by a porous barrier 208. A sampling port 209 is provided in the housing 202, in this implementation in an end wall of the housing 202, that provides access to the vapor space compartment 206. The sampling port 209 is covered by a sealing membrane 210. Prior to use of the calibration cartridge 200, the housing 202 is sealed (e.g., a hermetic seal) so internal fluids cannot escape the housing 202 and external fluids cannot enter the housing 202.

The calibration medium compartment 204 contains a precise ratio of a carrier fluid and a volatile substance, i.e., a working fluid, existing in both liquid and gaseous phases as a saturated fluid within the housing 202. The liquid-phase working fluid is stored in a gelatinous form in the calibration medium compartment 204 with the addition of a thickening or gelling agent. Any known thickening or gelling agent may be used that does not react with or dissipate in the presence of the working fluid. The porous barrier 208 allows passage of vapor phase working fluid to pass from the calibration medium compartment 204 to the vapor space 206 while inhibiting the passage of the gelled working fluid. In some implementations, a partial wall or barrier or merely a detent may be present to retain the gelled working fluid in the calibration medium compartment 204. In other implementations, the gelling agent is sufficient to confine the liquid-phase working fluid to the calibration medium compartment 204 without the use of the porous barrier 208.

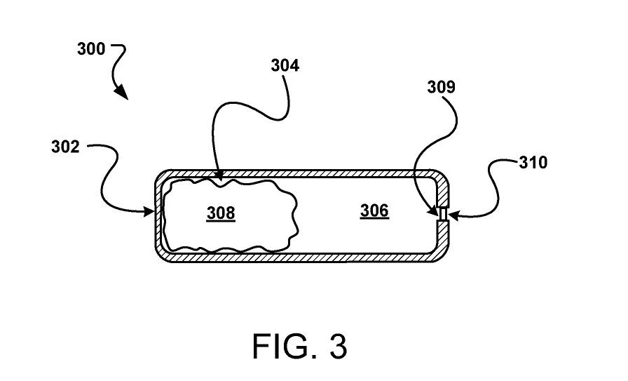

FIG. 3 illustrates another example disposable calibration cartridge 300. Various elements and features of cartridge 300 are the same or similar to like elements and features of cartridge 100 of FIG. 1, unless indicated otherwise.

The cartridge 300 includes a housing 302 that encloses a calibration medium compartment 304 and a vapor space compartment 306. A sampling port 309 is provided in the housing 302 that provides access to the vapor space compartment 306. The sampling port 309 is covered by a sealing membrane 310; in this implementation, the sealing membrane 310 is a thinner region of the housing 302. Prior to use of the calibration cartridge 300, the housing 302 is sealed (e.g., hermetically sealed) so internal fluids cannot escape the housing 302 and external fluids cannot enter the housing 302.

As in the previous implementations, the calibration medium compartment 304 contains a precise ratio of a carrier fluid and a volatile substance, i.e., a working fluid, existing in both liquid and gaseous phases as a saturated fluid within the housing 302. In this implementation, present within the calibration medium compartment 304 is an absorbent material 308 (e.g., a sponge or other porous solid object). The material 308 keeps liquid-phase working fluid within the calibration medium compartment 304 and away from the vapor space compartment 306. The material 308 may be adhesively or otherwise attached to the housing 302 to retain the material 308 in the calibration medium compartment 304 and away from the vapor space compartment 306. In other implementations, a physical barrier, such as a perforated barrier similar to barrier 208 of FIG. 2, may also be utilized to keep the material 308 in the calibration medium compartment 304.

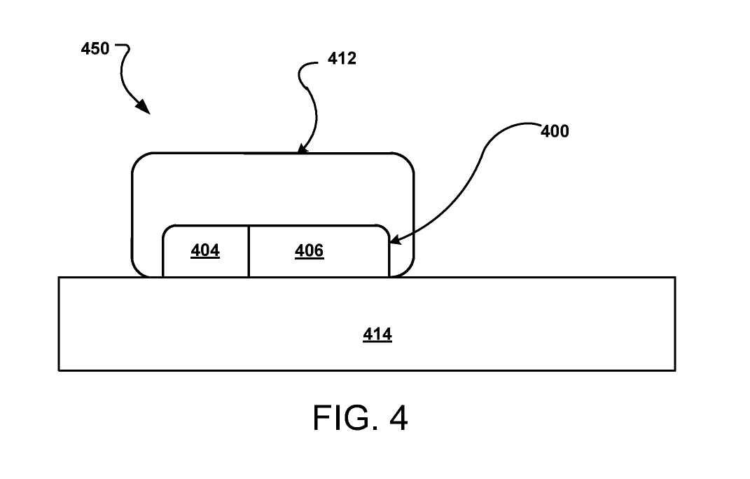

FIG. 4 illustrates an example disposable calibration cartridge 400 mounted to or in a cartridge heater 412. The disposable calibration cartridge 400 stores a working fluid in a calibration medium compartment 404 and a vapor space compartment 406 as described above with regard to the various disposable calibration cartridges described above. In this particular illustration, the cartridge 400 is similar to cartridge 100 of FIG. 1. The calibration cartridge 400 and the cartridge heater 412 are depicted on a table 414 for illustration purposes. The heater 412 and the cartridge 400 are collectively referred to herein as a calibration device 450.

Since the working fluid is stored at liquid-vapor equilibrium within the calibration cartridge 400, the temperature at which the working fluid is stored substantially affects the proportion of vaporized carrier fluid to vaporized alcohol within the cartridge 400. The cartridge 400 should be within 1 degree Celsius, or within 0.5 degrees Celsius, or within 0.1 degrees Celsius of a predetermined desired temperature in order to provide sufficiently accurate results. As a result, the disposable calibration cartridge 400 is most accurate for calibration when it is at the predetermined desired temperature (e.g., 10 to 200 degrees Celsius) where the proportion of vaporized carrier fluid to vaporized alcohol within the cartridge 400 is known.

In one example implementation, the predetermined desired temperature is at or near the temperature of a user's breath (e.g., 34 to 38 degrees Celsius). Since room temperature (e.g., 20 to 26 degrees Celsius) is lower than the predetermined desired temperature, the cartridge heater 412 is supplied to heat and maintain the calibration cartridge 400 at the predetermined desired temperature, for example, within 1 degree Celsius, or within 0.5 degrees Celsius, or within 0.1 degrees Celsius of the predetermined desired temperature. In other implementations, room temperature is higher than the predetermined desired temperature. As a result, the heater 412 is instead (or in addition) a cooler. In still further implementations, ambient temperature varies widely or is unknown. As a result, the heater 412 may function as both a heater and a cooler depending on whether the cartridge 400 needs to be heated or cooled to achieve the predetermined desired temperature.

In one particular example, the heater 412 is able to heat or cool the cartridge 400 to the desired temperature within 2 minutes, in some implementations within 1½ minutes, and in other implementations within 1 minute.

In another implementation, the heater 412 is replaced with a temperature detector. The temperature detector detects the temperature of the cartridge 400 and applies one of a number of calibration tables stored within the detector based on the cartridge 400 temperature. As a result, the cartridge 400 is not heated or cooled to a desired temperature but rather the calibration is adjusted based on the actual cartridge 400 temperature. In one implementation, the temperature detector connects to a temperature sensor (not shown) located inside the cartridge 400 to obtain an accurate temperature reading of the working fluid within the cartridge 400, in either or both the calibration medium compartment 404 and the vapor space compartment 406. In further implementations, a combination heater, cooler, and/or detector may be used to achieve a desired calibration accuracy.

The cartridge 400 fits or locks in place on or within the heater 412. In some implementations, the heater 412 can store and/or heat multiple cartridges 400 simultaneously. An interface between the heater 412 and the cartridge 400 enables heat transfer via thermal conduction, convection, and/or radiation from the heater 412 to the cartridge 400. Further, the cartridge 400 may be made of thermally conductive material that effectively transfers the heat generated by the heater 412 to the working fluid stored within the cartridge 400. In other implementations, a heating element (not shown) is located inside the cartridge 400 and is merely powered by the heater 412.

In some implementations, the heater 412 includes a mechanism for monitoring the cartridge 400 temperature and a feedback mechanism for changing the output from heater 412 based on the temperature of the cartridge 400 (e.g., turning the heater on when the cartridge 400 temperature is lower than the predetermined desired temperature and turning the heater 412 off when the cartridge 400 is at the predetermined desired temperature). In other implementations, the heater 412 maintains the predetermined desired temperature and the cartridge 400 is left in the heater 412 for a sufficient period of time to come to a relative temperature equilibrium with the heater 412. The heater 412 may include a visual and/or audio alarm to the user when the cartridge 400 achieves the predetermined desired temperature indicating that the cartridge 400 is ready or not ready for use.

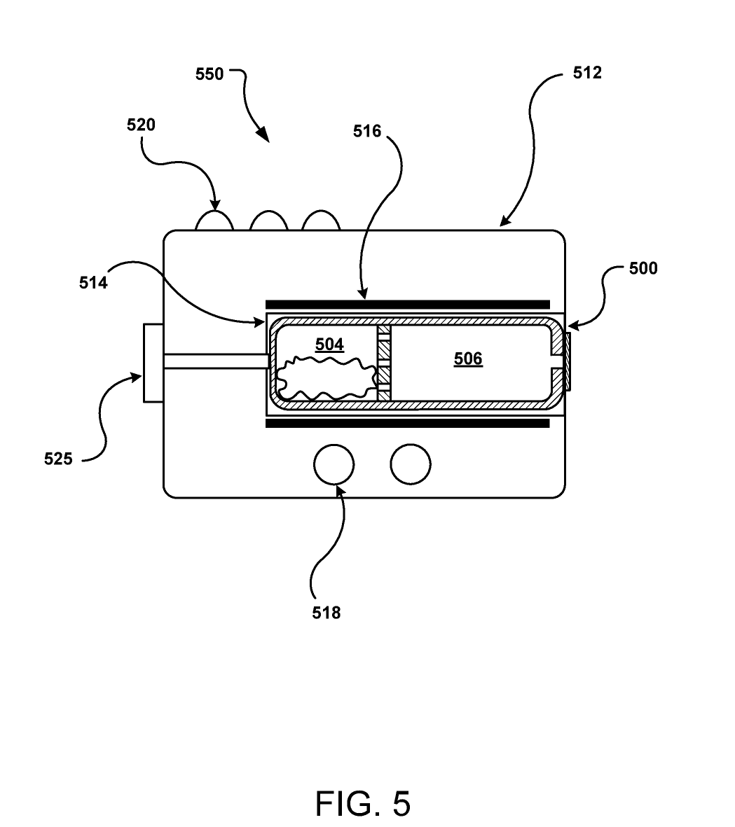

FIG. 5 illustrates another example disposable calibration cartridge 500 mounted to or in a cartridge heater 512. Various elements and features of cartridge 500 and heater 512 are the same or similar to like elements and features of cartridge 400 and heater 412 of FIG. 4, unless indicated otherwise.

As described above, the disposable calibration cartridge 500 stores a working fluid in a calibration medium compartment 504 and a vapor space compartment 506 as described above with regard to the various disposable calibration cartridges described above. In this particular illustration, the cartridge 500 is similar to cartridge 200 of FIG. 2. The heater 512 and the cartridge 500 are collectively referred to herein as a calibration device 550.

The heater 512 can be shaped and sized, and otherwise configured, to readily fit within the palm of a user's hand. Example dimensions include a height, width and thickness less than 10 cm in each dimension. The heater 512 may have a volume of 100 to 200 cubic cm. In one particular example, the heater 512 has approximate dimensions of 8 cm×6 cm×3 cm.

The cartridge 500 fits or locks within a receptacle or slot 514 in the heater 512. The cartridge 500 may include a visual indicator (e.g., indicia such as an arrow) indicating which end of the cartridge 514 should face out from the receptacle 514. Present, in this implementation, on two sides of the receptacle 514 are heater plates 516 powered by batteries 518 (e.g., rechargeable batteries, e.g., Li ion). In other implementations, heating mechanism(s) may be present on all sides of the receptacle 514. An interface between the heater 512 and the cartridge 500 enables heat transfer via thermal conduction, convection, and/or radiation from the heater 512 to the cartridge 500. Indicator lights 520 provide notification of, for example, when the cartridge 500 is properly inserted into the receptacle 514, when the heater plates 516 are activated, when the cartridge 500 has reached the desired temperature, if the batteries 518 are low, etc. Not shown, the heater 512 can additionally or alternately include an audible alarm, for example, for when the cartridge 500 is properly inserted into the receptacle 514, when the cartridge 500 has reached the desired temperature, if the batteries 518 are low, etc. The heater 512 may serve as a handle for manipulating and using the cartridge 500 to calibrate a breath alcohol tester.

The heater 512 can include an ejector mechanism 525 to eject the cartridge 500 from the receptacle 514 after the cartridge 500 has obtained the desired temperature. In various implementations, the cartridge 500 is ejected from the heater 512 after the cartridge 500 has been used to calibrate a device, such as a breath alcohol detector.

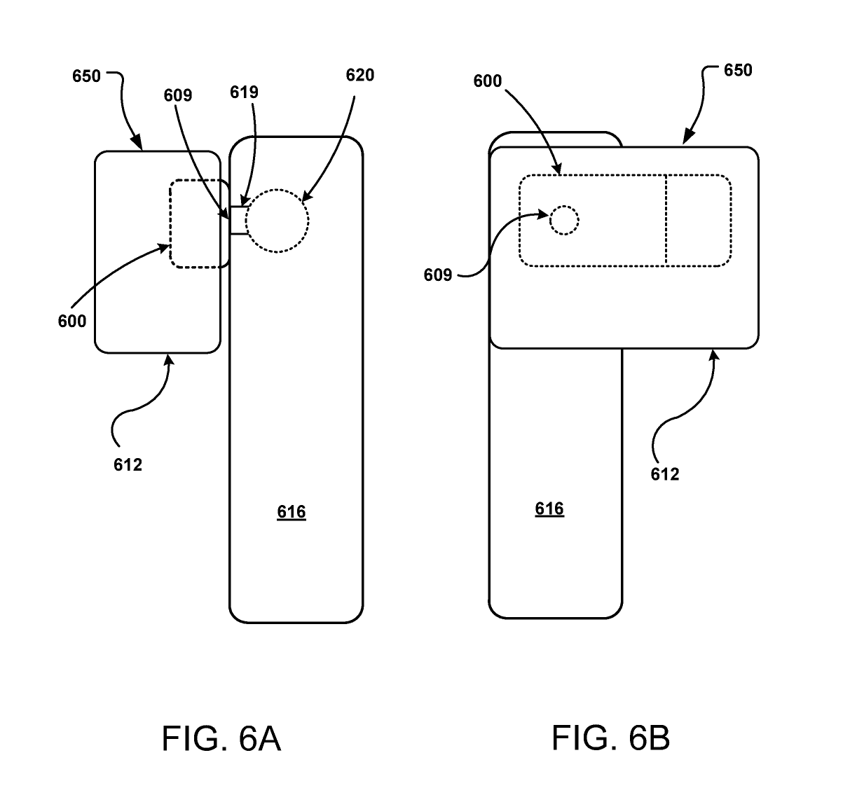

FIGS. 6A and 6B illustrate an example disposable calibration cartridge 600 mounted to a cartridge heater 612 (collectively a calibration device 650) being used to calibrate a breath alcohol tester 616. The heater 612 facilitates positioning the cartridge 600 to calibrate the breath alcohol tester 616.

The breath alcohol tester 616 can be any suitable breath alcohol tester (e.g., a fuel cell alcohol sensor or a semiconductor alcohol sensor) that has a sampling port for attaching a calibration standard. The cartridge heater 612 and the calibration cartridge 600 are as described above with regard to cartridge heater 412 of FIG. 4 and calibration cartridge 100 of FIG. 1, respectively. The disposable calibration cartridge 600 stores a working fluid as described above with regard to the various disposable calibration cartridges described above.

When a calibration operation (either a re-calibration or a calibration check) is performed on the breath alcohol tester 616, the calibration device 650 is oriented with a sampling port 609 of the cartridge 600 oriented in-line with a sampling port 619 of the breath alcohol tester 616. When the calibration device 650 is pressed against the breath alcohol tester 616, a puncturing device (not shown) ruptures the sealing membrane (not shown, see sealing membrane 109 of FIG. 1) and the sampling port of the breath alcohol tester 616 is exposed to gaseous working fluid from within the cartridge 600 of the calibration device 650. The breath alcohol tester 616 is thus calibrated (or merely calibration checked) using the gaseous working fluid from the calibration device 650.

In some implementations, the breath alcohol tester 616 includes a fan or pump 620 to draw the working fluid from within the calibration cartridge 600 into the breath alcohol tester 616. In other implementations, the calibration cartridge 600 or heater 612 (e.g., the calibration device 650) includes a plunger, pump, fan, or other device that pushes the working fluid from within the calibration cartridge 600 into the breath alcohol tester 616 or past the sampling port 619 of the breath alcohol tester 616. In other implementations, the breath alcohol tester 616 includes a pump, fan, or other device to draw the working fluid from within the calibration cartridge 600 into the breath alcohol tester 616.

In various implementations, the puncturing device may be a component of the breath alcohol tester 616 or the calibration device 650. Further, the puncturing device may be a sufficiently sharp and rigid needle or port that presses through the sealing membrane 610 or body of the calibration cartridge 600 (e.g., such as membrane 310 of cartridge 300 in FIG. 3); depending on the thickness, composition, strength, etc. of the sealing membrane 610, the puncturing device may be a blunt component with no sharp edges. The puncturing device may alternately be a separate device oriented between the calibration cartridge 600 and the calibration device 620 that adapts the calibration cartridge 600 to the calibration device 650, or a separate device oriented between the calibration cartridge 600 and the breath alcohol tester 616. In other implementations, the sealing membrane 610 slides open or peels off when the breath alcohol tester 616 is mounted to the calibration cartridge 600.

The breath alcohol tester 616 is calibrated (or merely calibration checked) using the gaseous working fluid within the calibration device 650. Once the calibration operation is complete, the breath alcohol tester 616 is removed from the calibration device 650 and the used calibration cartridge 600 is discarded. In some implementations, the cartridge 600 has a “use indicator,” to visually indicate that the cartridge 600 has been used; examples of such an indicator include a color change (e.g., due to contact pressure from the breath alcohol tester 616, due to exposure to ambient air, etc.). In some implementations, the puncture hole in the sealing membrane 610 is sufficient visual indication. The next calibration operation is performed using a new calibration cartridge mounted within the cartridge heater 612.

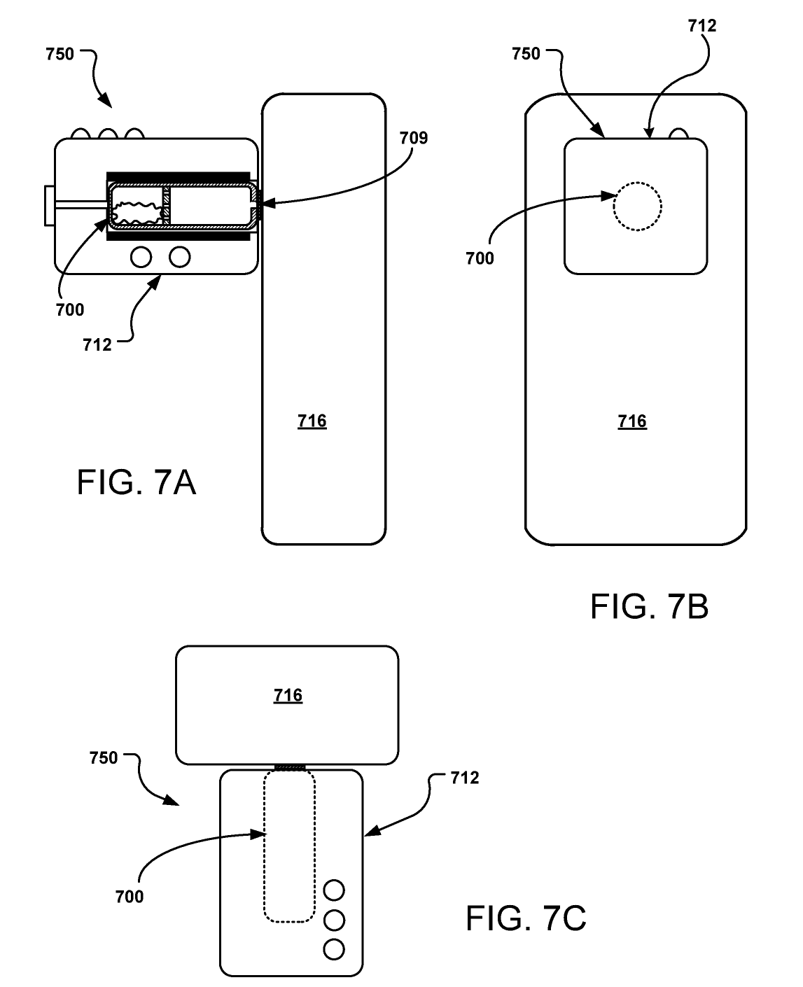

FIGS. 7A, 7B, 7C illustrate another example disposable calibration cartridge 700 mounted to a cartridge heater 712 (collectively a calibration device 750) being used to calibrate a breath alcohol tester 716. Various elements and features of the calibration device 750 and breath alcohol tester 716 are the same or similar to like elements and features of the calibration device 650 and breath alcohol tester 616 of FIG. 6, unless indicated otherwise. In this particular illustration, the cartridge 700 is similar to cartridge 200 of FIG. 2 and the heater 712 is similar to the heater 512 of FIG. 5.

The disposable calibration cartridge 700 stores a working fluid as described above with regard to the various disposable calibration cartridges described above, and the heater 712 heats and/or cools the cartridge 700 to a desired temperature. When a calibration operation (either a re-calibration or a calibration check) is performed on the breath alcohol tester 716, the calibration device 750 is oriented with a sampling port 709 of the cartridge 700 operably oriented with a sampling port of the breath alcohol tester 716. When the calibration device 750 is pressed against the breath alcohol tester 716, a puncturing device (not shown) ruptures the sealing membrane (not shown, see sealing membrane 209 of FIG. 2). As indicated above, the puncturing device may be a component of the breath alcohol tester 716 or the calibration device 750 or may be a separate device. The sampling port of the breath alcohol tester 716 is exposed to gaseous working fluid from within the cartridge 700 and the breath alcohol tester 716 is thus calibrated (or merely calibration checked) using the gaseous working fluid from the calibration device 750.

Once the calibration operation is complete, the breath alcohol tester 716 is removed from the calibration device 750 and the used calibration cartridge 700 is discarded. A new calibration cartridge can be mounted within the cartridge heater 712, brought to temperature, and used for a subsequent calibration operation.

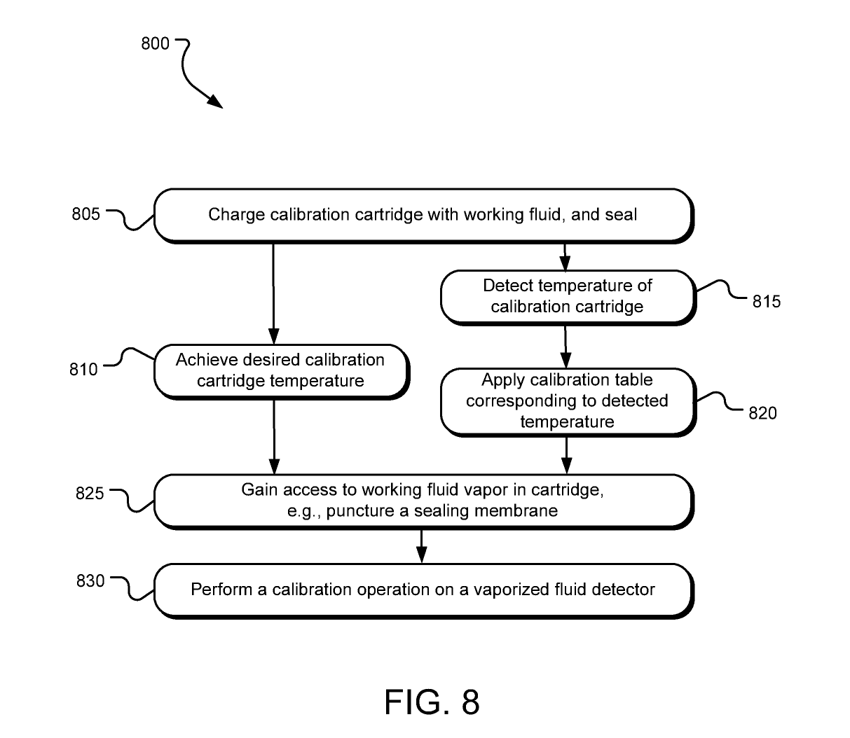

FIG. 8 illustrates example operations 800 for using a disposable calibration cartridge to calibrate a vaporized fluid detector, such as a breath alcohol tester. A charging operation 805 charges a calibration cartridge with a working fluid that is a combination of a known volatile compound to be detected and a carrier fluid. In an example implementation, the known volatile compound to be detected is ethanol and the carrier fluid is water. The working fluid is stored within the calibration cartridge in both liquid and gaseous phases. After charging the cartridge, the cartridge is sealed (e.g., hermetically sealed).

An achieving operation 810 achieves a desired calibration cartridge temperature. The ratio of the vaporized volatile compound to the carrier fluid may vary widely depending on temperature. As a result, the calibration cartridge is heated and/or cooled to achieve the desired calibration cartridge temperature; the ratio of the vaporized volatile compound to the carrier fluid is then known with a high degree of accuracy. Achieving operation 810 may be achieved using a heater and/or a cooler.

In another implementation, the operations 815, 820 are performed in lieu of achieving operation 810. Detection operation 815 detects a calibration cartridge temperature. In various implementations, the detection operation 815 is performed by a temperature sensor placed within or in close proximity to the calibration cartridge during the charging operation 805 or a temperature sensor inserted into the calibration cartridge during the accessing operation 825 (discussed below).

Applying operation 820 applies a calibration table corresponding to the detected calibration cartridge temperature. A heater, calibration device, or volatile substance detector stores a number of calibration tables and applies a selected calibration table that most closely matches the detected calibration cartridge temperature. In other implementations, the operations 810, 815, 820 are all performed to both modify the calibration cartridge temperature and select a calibration table that matches the modified calibration cartridge temperature.

An accessing operation 825 accesses the vapor phase working fluid in the calibration cartridge; in some implementations, this accessing is done by puncturing a sealing membrane on the calibration cartridge. When the calibration cartridge is ready to be used, it is interfaced with a volatile substance detector (e.g., a breath alcohol detector). As an example, a puncturing device located on the detector, the calibration cartridge, the heater, or other component oriented between the detector and the calibration cartridge punctures the sealing membrane, opening a port into the vapor for performing a calibration operation on the detector.

A performing operation 830 performs a calibration operation on the volatile substance detector. The calibration device takes a sample of the working fluid (e.g., vapor phase working fluid) within the calibration cartridge and uses the sample to either calibrate the detector or check an existing calibration of the detector for error. The accessing operation 825 and the performing operation 830 may be performed in quick succession (e.g., less than 1 second, e.g., less than 0.5 second) in order to prevent the working fluid within the punctured calibration cartridge from being contaminated by external contaminants or from being diluted.

The volatile or non-volatile substance that the above systems and methods are used to detect may be a variety of compounds or elements that can exist in a liquid-phase/gaseous-phase equilibrium at atmospheric pressure (e.g., ethanol, water, etc.). Examples of volatile substances that can be detected include alcohols (e.g., methanol, ethanol, isopropanol), ketones (e.g., acetone), paraffins, gasoline, volatile hydrocarbons (e.g., hexane), and any liquid that has a boiling point close to ambient temperature and pressure.

The logical operations making up the embodiments of the invention described herein are referred to variously as operations, steps, objects, or modules. Furthermore, it should be understood that logical operations may be performed in any order, adding or omitting operations as desired, unless explicitly claimed otherwise or a specific order is inherently necessitated by the claim language.

The above specification and examples provide a complete description of the structure and use of exemplary implementations of the invention. The above description provides specific implementations. It is to be understood that other implementations are contemplated and may be made without departing from the scope or spirit of the present disclosure. The above detailed description, therefore, is not to be taken in a limiting sense. While the present disclosure is not so limited, an appreciation of various aspects of the disclosure will be gained through a discussion of the examples provided.

Unless otherwise indicated, all numbers expressing feature sizes, amounts, and physical properties are to be understood as being modified by the term “about.” Accordingly, unless indicated to the contrary, the numerical parameters set forth are approximations that can vary depending upon the desired properties sought to be obtained by those skilled in the art utilizing the teachings disclosed herein.

As used herein, the singular forms “a”, “an”, and “the” encompass implementations having plural referents, unless the content clearly dictates otherwise. As used in this specification and the appended claims, the term “or” is generally employed in its sense including “and/or” unless the content clearly dictates otherwise.

Spatially related terms, including but not limited to, “bottom,” “lower”, “top”, “upper”, “beneath”, “below”, “above”, “on top”, “on,” etc., if used herein, are utilized for ease of description to describe spatial relationships of an element(s) to another. Such spatially related terms encompass different orientations of the device in addition to the particular orientations depicted in the figures and described herein. For example, if a structure depicted in the figures is turned over or flipped over, portions previously described as below or beneath other elements would then be above or over those other elements.

Since many implementations of the invention can be made without departing from the spirit and scope of the invention, the invention resides in the claims hereinafter appended. Furthermore, structural features of the different implementations may be combined in yet another implementation without departing from the recited claims.

1. A calibration cartridge comprising:
a housing having an interior volume;
a saturated working fluid including a detected substance and a carrier fluid stored within the interior volume in both a liquid phase and a gaseous phase;
a solid absorbent medium retaining the liquid phase working fluid within the interior volume; and
a sealing membrane on the housing forming a hermetically sealed interior volume.

2. The calibration cartridge of claim 1, wherein the detected substance is ethanol and the carrier fluid is water.

3. The calibration cartridge of claim 1, wherein the saturated working fluid is at a known ratio of the detected substance to the carrier fluid.

4. The calibration cartridge of claim 1, wherein the saturated working fluid has 25-1050 ppm ethanol to water.

5. The calibration cartridge of claim 1, wherein the housing includes:
a calibration medium compartment that stores the saturated working fluid in the liquid phase and the gaseous phase; and
a vapor space compartment that stores the saturated working fluid in the gaseous phase only.

6. The calibration cartridge of claim 5, further comprising a permeable barrier that prevents transmission of the liquid phase saturated working fluid from the calibration medium compartment to the vapor space compartment and permits transmission of gaseous phase saturated working fluid between the calibration medium compartment and the vapor space compartment.

7. (canceled)

8. A calibration cartridge comprising:
a sealed housing having a vapor space compartment and a calibration medium compartment;
a working fluid present in a gaseous phase in the vapor space compartment, and the working fluid present in both a gaseous phase and a liquid phase in the calibration medium compartment;
a solid absorbent medium retaining the liquid chase working fluid within the calibration medium compartment; and
an access port through the housing into the vapor space compartment.

9. The calibration cartridge of claim 8, further comprising a gas-permeable and liquid non-permeable barrier between the calibration medium compartment and the vapor space compartment.

10. (canceled)

11. The calibration cartridge of claim 8, wherein the solid absorbent medium retaining the liquid phase working fluid is a gel.

12. The calibration cartridge of claim 8, wherein the access port has a sealing membrane thereover.

13. The calibration cartridge of claim 8, wherein the access port is defined by a thinner wall region of the housing.

14. The calibration cartridge of claim 8, wherein the vapor space compartment and the calibration medium compartment have a combined volume of 1-10 cubic cm.

15. The calibration cartridge of claim 8, wherein the working fluid comprises water and ethanol.

16. The calibration cartridge of claim 15, wherein the ethanol is present at 5-1300 ppm in the water.

17. A method of calibrating a vaporized fluid detector comprising:
charging a calibration cartridge with a solid absorbent medium retaining a saturated working fluid, the saturated working fluid including a detected substance and a carrier fluid;
accessing a saturated working fluid vapor in the calibration cartridge; and
performing a calibration operation on the vaporized fluid detector using the saturated working fluid vapor within the calibration cartridge.

18. The method of claim 17, further comprising:
heating the calibration cartridge to a predetermined desired temperature prior to accessing the saturated working fluid vapor.

19. The method of claim 17 comprising:
rupturing a sealing membrane on the calibration cartridge to access the saturated working fluid vapor.

20. The method of claim 17 comprising:
rupturing a region of a wall of the calibration cartridge to access the saturated working fluid vapor.

21. The calibration cartridge of claim 1, wherein the sealing membrane is configured to be ruptured to access the interior volume of the housing.

22. The calibration cartridge of claim 12, wherein the sealing membrane is configured to be ruptured to access the vapor space compartment of the sealed housing.
